(19) United States
(12) Patent Application Publication
(10) Pub. No.: US 2023/0414758 A1
(43) Pub. Date: Dec. 28, 2023

(54) CAPSAICIN-DERIVED PHOTOSENSITIZER, AND PREPARATION METHOD AND USE THEREOF

(71) Applicant: SOOCHOW UNIVERSITY, Suzhou (CN)

(72) Inventors: Zhengqing GUO, Suzhou (CN); Hui HE, Suzhou (CN); Han XU, Suzhou (CN); Yangyang HUANG, Suzhou (CN); Dandan JI, Suzhou (CN); Guanglin WANG, Suzhou (CN); Kewang BAO, Suzhou (CN); Guoqing CUI, Suzhou (CN)

(21) Appl. No.: 18/196,929

(22) Filed: May 12, 2023

Related U.S. Application Data
(63) Continuation of application No. PCT/CN2022/114483, filed on Aug. 24, 2022.

(30) Foreign Application Priority Data
Jun. 22, 2022 (CN) 202210712493.6

(51) Int. Cl. A61K 41/00; B82Y 5/00

(52) U.S. Cl. CPC A61K 41/0057; B82Y 5/00; A61K 2121/00

(57) ABSTRACT

The present invention discloses a capsaicin-derived photosensitizer, and a preparation method and use thereof. Boron-dipyrromethene is used as a core of the photosensitizer, which has a maximum absorption peak red-shifted to the near infrared region by expanding the π system and is introduced with a capsaicin-targeting group to construct a capsaicin-derived photosensitizer cap-BDP with high-performance targeting of TRPV1 protein. The photosensitizer can generate singlet oxygen under low-power LED irradiation, which specifically activates the TRPV1 protein pathway, causes significant calcium influx, and induces apoptosis of tumor cells. Moreover, the generated singlet oxygen directly oxidizes endogenous substances in cells to cause cell apoptosis, eventually achieving light-induced bifunctional synergistic therapy. Also, a nano-photosensitizer cap-BDP-NPs is assembled in an aqueous media by using amphiphilic block polymers as drug carriers of photosensitizer cap-BDP, which can be applied in the light-induced treatment of triple negative breast cancer.

### FIELD OF THE INVENTION

The present invention relates to the technical field of nano-materials and biomedicines, and particularly to a capsaicin-derived photosensitizer, and a preparation method and use thereof.

### DESCRIPTION OF THE RELATED ART

TRPV1 protein was successfully cloned and named by Professor Julius, winner of the 2021 Nobel Prize in Physiology, and his team at the end of the 20th century. Studies show that TRPV1 protein can be specifically activated by capsaicin or heat (>43° C.). These physical stimuli can be uniformly converted into an electrical signal through the TRPV1 channel on the cell membrane, which is successively transmitted between nerve cells, to eventually reach the cerebral cortex and produce different sensations. This finding not only explains the irritating sensation felt when peppers are ingested, but also reveals the biological mechanism that a human senses the temperature. After TRPV1 protein is activated, Ca²⁺ influx and K⁺ efflux are caused, to regulate the cellular calcium signaling or membrane depolarization, mediate the response in the body to endogenous and exogenous chemical stimuli and physical stimuli such as temperature, and transmit relevant signals to the central nervous system and produce the pain sensation. In addition to capsaicin, vanillin derivatives, heat (>43° C.), and acidity (pH<6), TRPV1 protein can be activated by arachidonic acid metabolites, hydrogen peroxide, nitric oxide, active oxygen species and other endogenous substances in the body, to shows rich biological effects. Attempts are made by many pharmaceutical companies to develop TRPV1 targeted drugs. Capsaicin and Zucapsaicin are approved for the treatment of osteoarthritis, herpes zoster-induced neuralgia, and diabetic peripheral neuralgia. There are still more than 20 TRPV1 targeted drugs in the research and development stage.

Moreover, in recent years, studies show that TRPV1 protein plays an important role in the occurrence and development of breast cancer, endometrial cancer, prostate cancer, bladder cancer, melanoma, liver cancer and other types of tumors, and directly correlates with the tumor cell proliferation, death and metastasis. Capsaicin is used by the researchers to act on TRPV1 protein of intestinal epithelial cells to activate calpain, causing the activation of protein tyrosine phosphatase 1B, thereby inhibiting the proliferation of epithelial cells induced by EGFR, and finally inhibiting the occurrence of intestinal tumors. Other data shows that TRPV1 agonists can effectively activate Ca²⁺ channels of TRPV1 protein, causing Ca²⁺ influx and eventually inducing the tumor cell death. However, TRPV1 protein is widely distributed in many organs, tissues and cells in human body, and shows complex physiological and pathological functions. However, the current TRPV1 agonists generally have poor water solubility, insufficient performance of targeting, high dosage and others, causing serious toxic and side effects.

Based on the thermal sensitivity of TRPV1 protein, a semiconductor nano-polymer SPN with high photothermal conversion efficiency is designed and synthesized by Pu project team by nano-delivery technology, which can precisely control the neuronal TRPV1 protein activation by photothermal effect (J. Am. Chem. Soc. 2016, 138, 9049-9052). SPN shows high photothermal conversion ability, can specifically bind to temperature-sensitive TRPV1 protein, and quickly activate the Ca²⁺ influx of nerve cells in a safe and reversible way. Then, on the basis of the SPN compound, capsaicin (CAP) is encapsulated by the Pu project team to form an ion channel targeted nano-drug SPN-C, which can activate TRPV1 protein by photothermal effect (Nano Lett, 2018, 18, 1498-1505). Through multiple irradiations in a short time, the nano-micelle can repeatedly release the TRPV1 agonist CAP, to activate the TRPV1 channel on the cell membrane in multiple ways, causing a cumulative effect and inducing the apoptosis. A nano-micelle for activating TRPV1 protein by photothermal effect is designed and synthesized by another researcher (iScience, 2020, 23, 101049). CuS with high photothermal conversion rate is encapsulated in a CaCO₃ nano-micelle, and the biocompatibility is improved by modification with polyethylene glycol. Finally, CuS@CaCO₃-PEG nano-micelle is obtained. CuS@CaCO₃-PEG is passively targeted to tumor cells by means of EPR effect, and responsively decomposed in an acidic tumor microenvironment, to release CuS and Ca²⁺. Under irradiation conditions, the high heat generated by CuS nano-particles activates TRPV1 protein, so Ca′ influx into the cells occurs and the concentration of intracellular calcium is rapidly increased, causing mitochondrial dysfunction (up-regulation of Caspase-3 and Cytochrome c and down-regulation of Bcl-2 and ATP) to kill the cells. Although the photothermal effect shows good TRPV1 protein activating performance, this process often requires a high concentration of a photothermal reagent to target the tumor tissues. Moreover, the transfer of high heat depends heavily on the heat exchange efficiency of the surrounding medium, the accuracy is poor and difficult to control, causing damage to surrounding normal tissues or cells.

Therefore, how to improve the performance of targeting of the TRPV1 agonists to achieve the accurate activation of TRPV1 protein is a key problem to be solved urgently.

### SUMMARY OF THE INVENTION

In view of the above technical problems, the present invention provides a capsaicin-derived photosensitizer, and a preparation method and use thereof. Boron-dipyrromethene (BODIPY) is used as a core of the photosensitizer, which has a maximum absorption peak red-shifted to the near infrared region by expanding the π system and is introduced with a capsaicin-targeting group to construct a capsaicin-derived photosensitizer cap-BDP with high-performance targeting of TRPV1 protein. Under low-power LED light irradiation, light-induced activation of TRPV1 protein channel is achieved, and bifunctional synergistic therapy of tumors is realized by means of the photodynamic activity. A nano-photosensitizer formed by self-assembly of cap-BDP with amphiphilic block polymers is useful in the light-induced treatment of triple negative breast cancer.

To solve the above technical problems, the present invention provides the following technical solutions.

In a first aspect, the present invention provides a capsaicin-derived photosensitizer, having a general structural formula shown below:

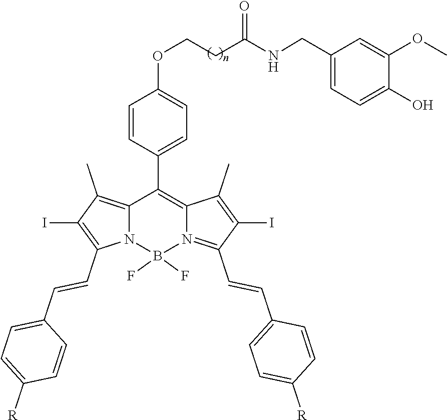

where n is any integer from 1 to 10; and
R is selected from hydrogen, C1-C8 alkyl, and C1-C8 alkoxy.

Further, R is hydrogen, methyl, ethyl, propyl, butyl pentyl, hexyl, octyl, methoxy, ethoxy, 4-propynyloxy or tert-butoxy.

In a second aspect, the present invention provides a method for preparing the capsaicin-derived photosensitizer according to the first aspect. The method includes the following steps: under an inert atmosphere,
(1) reacting Compound of Formula (I) with 2,4-dimethylpyrrole in the presence of trifluoroacetic acid and an organic solvent, then adding an oxidant for further reaction, adding boron trifluoride etherate and an organic amine to the system after the reaction, and reacting to obtain Compound of Formula (II);
(2) reacting Compound of Formula (II) with an iodination reagent in the presence of an organic solvent, to obtain Compound of Formula (III);
(3) reacting Compound of Formula (III) with Compound of Formula (IV) in the presence of acetic acid, piperidine and an organic solvent, to obtain Compound of Formula (V); and
(4) reacting Compound of Formula (V) with vanillylamine or a salt thereof in the presence of a condensing agent and an organic solvent, to obtain the capsaicin-derived photosensitizer.

The structures of Formulas (I)-(V) are shown below:

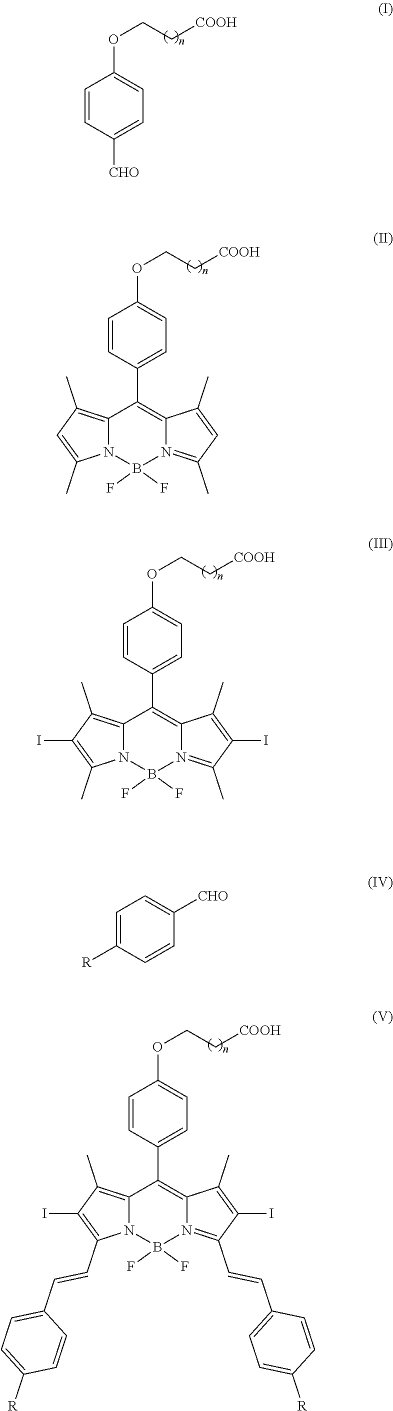

where n is any integer from 1 to 10, R is selected from hydrogen, C1-C8 alkyl, and C1-C8 alkoxy.

Further, under an inert atmosphere, the Compound of Formula (I) is obtained by reaction of p-hydroxybenzaldehyde with a haloalkylcarboxylic acid under reflux in the presence of an acid acceptor, a catalyst, and an organic solvent, where the haloalkylcarboxylic acid is BrCH₂(CH₂)ₙCOOH or ICH₂(CH₂)ₙCOOH, in which n is any integer from 1 to 10; the acid acceptor is potassium carbonate, sodium carbonate or cesium carbonate; the catalyst is benzo-18-crown-6-ether; and the organic solvent is preferably acetonitrile.

Further, in Step (1), the oxidant is 2,3-dichloro-5,6-dicyano-p-benzoquinone or selenium dioxide.

Further, in Step (1), boron trifluoride etherate and an organic amine are added to the system and reacted in an ice bath.

Further, in Step (1), the organic amine is triethyl amine or diisopropylethyl amine.

Further, in Step (1), the organic solvent is preferably tetrahydrofuran.

Further, in Step (2), the iodination reagent includes, but is not limited to, N-iodosuccinimide, elemental iodine, or N-iodosaccharin.

Further, in Step (2), the organic solvent is dichloromethane.

Further, in Step (2), the reaction is preferably carried out in the dark.

Further, in Step (3), the organic solvent is acetonitrile.

Further, in Step (3), the reaction temperature is 50 to 130° C., and the reaction time is not less than 0.5 hr.

Further, in Step (4), the salt of vanillylamine includes vanillylamine hydrochloride.

Further, in Step (4), the condensing agent is (1-cyano-2-ethoxy-2-oxoethylideneaminooxy)dimethylamino-morpholin-carbonium hexafluorophosphate, dicyclohexyl carbodiimide, 1-ethyl-(3-dimethylaminopropyl)carbodiimide hydrochloride or 2-(7-azabenzotriazole)-N,N,N′,N′-tetramethyluronium hexafluorophosphate.

Further, the organic solvent is N,N-dimethylformamide.

Further, in Step (4), the reaction is carried out in the presence of an acid acceptor and/or a catalyst, in which the acid acceptor is diisopropylethyl amine or triethyl amine, and the catalyst is 4-dimethylaminopyridine.

In a third aspect, the present invention provides a nano-photosensitizer, which is formed by self-assembly of the capsaicin-derived photosensitizer according to the first aspect with an amphiphilic block polymer in water.

Further, the amphiphilic block polymer is selected from the group consisting of polyethylene glycol-b-polycaprolactone, polyethylene glycol-polyglutamic acid, poly2-(diisopropylamino)ethyl methacrylate, and polyethylene glycol-polybenzyl glutamate.

In a fourth aspect, the present invention provides a method for preparing the nano-photosensitizer according to the third aspect. The method includes dissolving the capsaicin-derived photosensitizer according to the first aspect and an amphiphilic block polymer in an organic solvent, adding water to the obtained solution, and dialyzing to obtain the nano-photosensitizer; where the dialyzing medium for dialysis is water.

Further, water is added at a volume ratio to the solution of 1-10:1; and the particle size of nano-photosensitizer can be adjusted by adjusting the amount of water added.

In a fifth aspect, the present invention provides use of the nano-photosensitizer according to the third aspect in the preparation of drugs for light-induced treatment of triple negative breast cancer.

The present invention has the following beneficial effects. 1. In the present invention, boron-dipyrromethene (BODIPY) is used as a core that has a maximum absorption peak red-shifted to the near infrared region by expanding the π system and is introduced with a capsaicin-targeting group, to construct a TRPV1 targeted near-infrared photosensitizer cap-BDP. Under low-power LED light irradiation (660 nm, 20 mW cm⁻²), the photosensitizer cap-BDP shows a powerful singlet oxygen production ability, with a singlet quantum yield of 0.73. In addition, it is found through in-vitro cell experiments in the present invention that the photosensitizer cap-BDP can effectively activate TRPV1 protein and change the intracellular calcium concentration. Light irradiation can effectively improve the calcium regulation ability of the photosensitizer cap-BDP, and TRPV1 protein is further activated by singlet oxygen (¹O₂) to increase the intracellular calcium concentration, thus inducing apoptosis of tumor cells. Moreover, the generated singlet oxygen directly oxidizes endogenous substances in cells to kill tumor cells. Moreover, the present invention confirms that calcium influx is realized through TRPV1 channel at the cellular level.

2. A nano-photosensitizer cap-BDP-NPs is formed by assembly of an amphiphilic block polymer used as a drug carrier with the photosensitizer cap-BDP in an aqueous media in the present invention. The nano-photosensitizer prepared is uniform in size, and can be used to test in vivo biological effect. In the present invention, the pharmacokinetics and in vivo tumor inhibition of the nano-photosensitizer cap-BDP-NPs are determined through animal experiments. The test results show that the nano-photosensitizer cap-BDP-NPs has a good long circulation effect, which is conducive to the enrichment at tumor sites; and the nano-photosensitizer cap-BDP-NPs can effectively generate reactive oxygen species after illumination and cause the calcium influx at the tumor site in mice, so as to achieve a synergistic therapeutic effect. Therefore, the nano-photosensitizer can effectively inhibit tumors and even achieve the effect of ablation of some tumors, shows a superior in vivo therapeutic effect, and is expected to achieve the efficient treatment of triple negative breast cancer.

### BRIEF DESCRIPTION OF THE DRAWINGS

FIG. 1 shows the synthesis route of the photosensitizer cap-BDP;

FIG. 2 is a ¹H NMR spectrum of the photosensitizer cap-BDP prepared in Example 1;

FIG. 3 shows a UV-vis absorption spectrum and a fluorescence emission spectrum (FL) of the photosensitizer cap-BDP prepared in Example 1;

FIG. 4 shows a UV-vis absorption spectrum and a fluorescence emission spectrum (FL) of the nano-photosensitizer cap-BDP-NPs prepared in Example 7;

FIG. 5 shows diphenylisobenzofuran (DPBF) quenched by the photosensitizer cap-BDP prepared in Example 1 and Zinc phthalocyanine (ZnPc) under irradiation;

FIG. 6 is a fluorescence image showing the changes in intracellular calcium concentration before and after irradiation, scale 20 μm;

FIG. 7 is a statistical bar chart of fluorescence intensity showing the intracellular calcium concentration before and after irradiation;

FIG. 8 is a fluorescence image showing the inhibition of TRPV1 channel, scale 100 μm;

FIG. 9 shows the toxicity test of the photosensitizer BDP and cap-BDP on 4T1 cells with (left) or without (right) irradiation;

FIG. 10 is the phototoxicity test of the photosensitizers BDP and cap-BDP on triple negative breast cancer 4T1 cells in the presence of a calcium source;

FIG. 11 is a dynamic light scattering diagram of the nano-photosensitizer cap-BDP-NPs;

FIG. 12 is a transmission electron microscopy image of the nano-photosensitizer cap-BDP NPs, scale 200 nm;

FIG. 13 shows the in vivo circulation half-lives of the nano-photosensitizer 125 I-cap-BDP-NPs and photosensitizer 125 I-cap-BDP;

FIG. 14 shows the tissue distribution of the nano-photosensitizer 125 I-cap-BDP-NPs and photosensitizer 125 I-cap-BDP;

FIG. 15 shows SPECT-CT images of the nano-photosensitizer 125 I-cap-BDP-NPs and photosensitizer 125 I-cap-BDP in mice;

FIG. 16 is an image showing the calcium at the tumor site, scale 200 μm; and

FIG. 17 shows the tumor change curves of mice in different groups.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

Unless otherwise stated, all technical and scientific terms used herein have the same meaning as commonly understood by persons skilled in the art to which the present invention pertains. The terms used in the descriptions of the present invention are for the purpose of describing specific embodiments only and are not intended to limit the present invention. The term “and/or” as used herein includes any and all combinations of one or more of the listed related items.

In the examples of the present invention, the structure of the compound is determined by nuclear magnetic resonance (NMR) or mass spectrometry (MS). The NMR spectrum is measured by Agilent 400 MHz or 600 MHz NMR instrument. The determination solvent is deuterated dimethyl sulfoxide (DMSO-d₆) and deuterated chloroform (CDCl₃), and the internal standard is tetramethylsilane (TMS). The MS spectrum is measured by GCT Premier™ (CI) MS spectrometer, with a CI source (70 ev) unless otherwise indicated.

### Example 1

This example involves the preparation of a compound having a structural formula below:

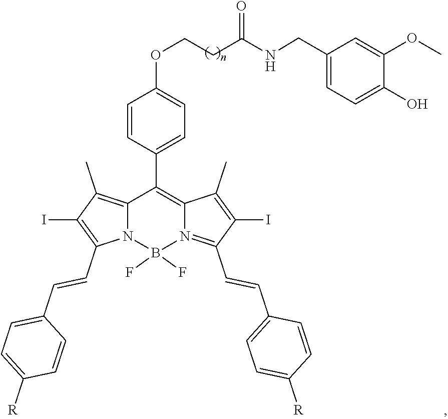

where n=4, and R is methoxy.

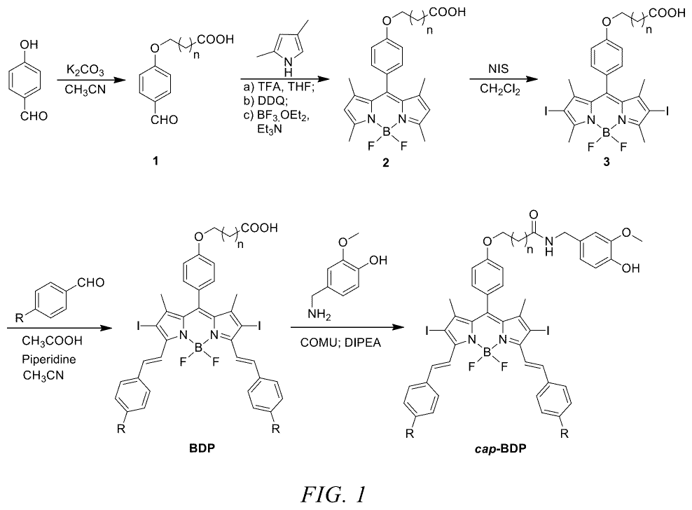

The compound is prepared through a synthesis route as shown in FIG. 1, and the process comprise specifically the following steps:

(1) Synthesis of Compound 1: p-hydroxybenzaldehyde, 6-bromohexanoic acid and potassium carbonate were weighed and added to a reactor at a molar ratio of 1:2:2. Then acetonitrile of 5 times the weight of 6-bromohexanoic acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with cold acetonitrile to obtain a white crude product. The white crude product was dissolved in ultrapure water, and then neutralized with 4 mol/L hydrochloric acid to obtain a white precipitate. The precipitate was freeze dried, to obtain Compound 1 (yield 80%).

(2) Synthesis of Compound 2: Compound 2,4-dimethylpyrrole and Compound 1 were weighed and added to a reactor at a molar ratio of 2:1. Then, anhydrous tetrahydrofuran of 10 times the weight of 2,4-dimethylpyrrole was added as a reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Tetrahydrofuran was removed by rotary evaporation, The residue was extracted with ethyl acetate, dried with anhydrous sodium sulfate and filtered. The organic solvent was removed by rotary evaporation, and the residue was purified by column chromatography on silica gel (eluent: petroleum ether and dichloromethane containing 1% acetic acid). Finally, an orange-red solid product 2 was obtained (yield 60%).

(3) Synthesis of Compound 3: Compound 2 and N-iodosuccinimide were weighed and added to a reactor at a molar ratio of 1:2. Anhydrous dichloromethane of 10 times the weight of N-iodosuccinimide was added as a solvent, and the reaction was continued for 30 hrs in the dark at normal temperature under argon atmosphere. After the reaction, dichloromethane was removed by rotary evaporation, and the residue was purified by column chromatography on silica gel (eluent: petroleum ether and dichloromethane containing 1% acetic acid). Finally, a brick red solid product 3 was obtained (yield 90%).

(4) Synthesis of photosensitizer BDP: Compound 3 and p-methoxybenzaldehyde were weighed and added to a reactor at a molar ratio of 1:1. Acetic acid and piperidine of 20 times the molar amount of p-methoxybenzaldehyde were added, and then acetonitrile of 20 times the weight of p-methoxybenzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: petroleum ether and dichloromethane containing 1% acetic acid). A blue green solid product BDP was obtained (yield 65%).

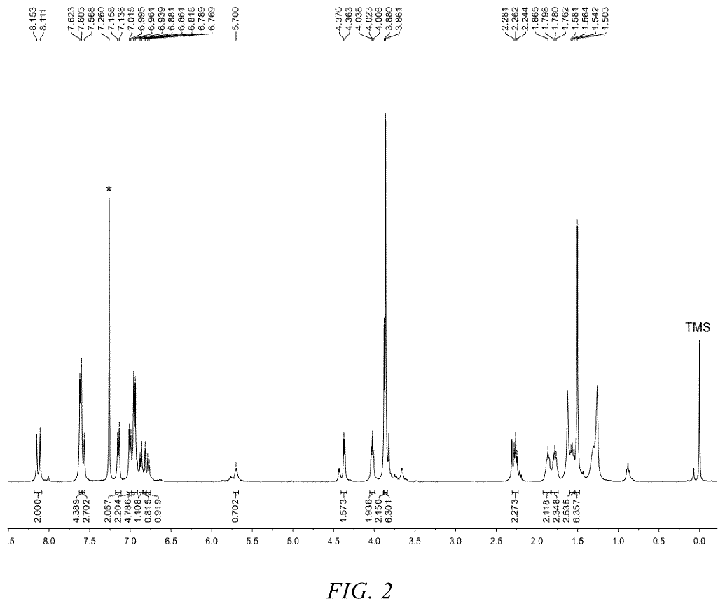

(5) Synthesis of photosensitizer cap-BDP: Compound diisopropylethyl amine and vanillylamine hydrochloride were weighed and added to reactor at a molar ratio of 3:2. N,N-dimethyl formamide of 5 times the weight of diisopropylethyl amine was added as a solvent, and stirred for 15 min at 45° C. under argon atmosphere. Then, the reaction system was positioned in ice bath, and cooled to 0° C. At this time, BDP of 0.5 time of the molar amount of diisopropylethyl amine was added to the reactor, and reacted for another 30 min with stirring. The condensing agent (1-cyano-2-ethoxy-2-oxoethylideneaminooxy)dimethylamino-morpholin-carbonium hexafluorophosphate of 2 times the molar amount of diisopropylethyl amine was slowly added to the reaction system, and reacted in an ice bath for another 30 hrs with stirring. After the reaction, N,N-dimethyl formamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: ethyl acetate and dichloromethane). A dark green product cap-BDP was obtained (yield 65%). The ¹H NMR spectrum of the photosensitizer cap-BDP is shown in FIG. 2.

### Example 2

This example involves the preparation of a compound having a structural formula below:

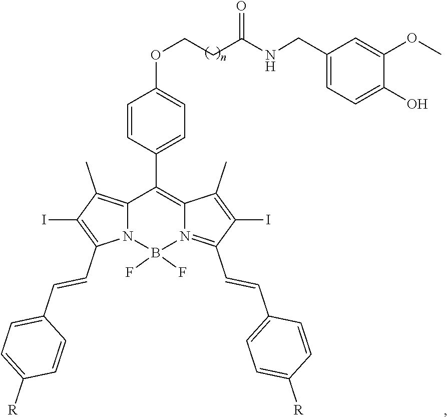

where n=0, and R is hydrogen.

The compound is prepared through a synthesis route as shown in FIG. 1, and the process comprises specifically the following steps:

(1) Synthesis of Compound 1: p-hydroxybenzaldehyde, 2-bromoacetic acid and potassium carbonate were weighed and added to a reactor at a molar ratio of 1:2:2. Then, acetonitrile of 10 times the weight of 2-bromoacetic acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with cold acetonitrile to obtain a white crude product. The white crude product was dissolved in ultrapure water, and then neutralized with 4 mol/L hydrochloric acid to obtain a white precipitate. The precipitate was freeze dried, to obtain Compound 1 (yield 70%).

(2) Synthesis of Compound 2: Compound 2,4-dimethylpyrrole and Compound 1 were weighed and added to a reactor at a molar ratio of 2:1. Then, anhydrous acetonitrile of 10 times the weight of 2,4-dimethylpyrrole was added as a reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Acetonitrile was removed by rotary evaporation. The residue was extracted with ethyl acetate, dried with anhydrous sodium sulfate and filtered. The organic solvent was removed by rotary evaporation, and the residue was purified by column chromatography on silica gel (eluent: petroleum ether and ethyl acetate containing 1% acetic acid). Finally, an orange-red solid product 2 was obtained (yield 70%).

(3) Synthesis of Compound 3: Compound 2 and N-iodosuccinimide were weighed and added to a reactor at a molar ratio of 1:2. Anhydrous tetrahydrofuran of times the weight of N-iodosuccinimide was added as a solvent, and the reaction was continued for 30 hrs in the dark at normal temperature under argon atmosphere. After the reaction, tetrahydrofuran was removed by rotary evaporation, and the residue was purified by column chromatography on silica gel (eluent: petroleum ether and dichloromethane containing 1% acetic acid). Finally, a brick red solid product 3 was obtained (yield 90%).

(4) Synthesis of photosensitizer BDP: Compound 3 and benzaldehyde were weighed and added to a reactor at a molar ratio of 1:1. Acetic acid and piperidine of times the molar amount of benzaldehyde were added, and then acetonitrile of 30 times the weight of benzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: petroleum ether and dichloromethane containing 1% acetic acid). A blue green solid product BDP was obtained (yield 65%).

(5) Synthesis of photosensitizer cap-BDP: Compound diisopropylethyl amine and vanillylamine hydrochloride were weighed and added to reactor at a molar ratio of 3:2. N,N-dimethyl formamide of 5 times the weight of diisopropylethyl amine was added as a solvent, and stirred for 15 min at 45° C. under argon atmosphere. Then, the reaction system was positioned in ice bath, and cooled to 0° C. At this time, BDP of time of the molar amount of diisopropylethyl amine was added to the reactor, and reacted for another 30 min with stirring. The condensing agent dicyclohexyl carbodiimide and 4-dimethylaminopyridine of 2 times the molar amount of diisopropylethyl amine were slowly added to the reaction system, and reacted in an ice bath for another 30 hrs with stirring. After the reaction, N,N-dimethylformamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: ethyl acetate and dichloromethane). A dark green product cap-BDP was obtained (yield 65%).

### Example 3

This example involves the preparation of a compound having a structural formula below:

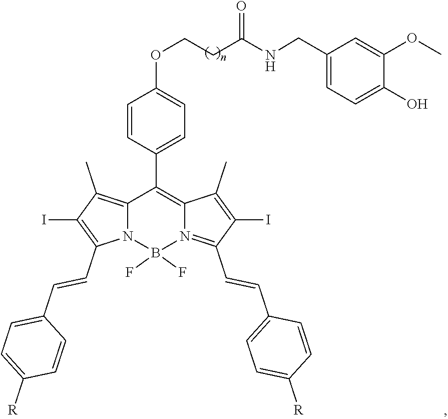

where n=2, and R is 4-propynyloxy.

The compound is prepared through a synthesis route as shown in FIG. 1, and the process comprises specifically the following steps:

(1) Synthesis of Compound 1: p-hydroxybenzaldehyde, 4-bromobutyric acid and sodium carbonate were weighed and added to a reactor at a molar ratio of 1:2:2. Then, acetonitrile of 10 times the weight of 4-bromobutyric acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with petroleum ether to obtain a white crude product. The white crude product was dissolved in ultrapure water, and then neutralized with 4 mol/L hydrochloric acid to obtain a white precipitate. The precipitate was freeze dried, to obtain Compound 1 (yield 64%).

(2) Synthesis of Compound 2: Compound 2,4-dimethylpyrrole and Compound 1 were weighed and added to a reactor at a molar ratio of 2:1. Anhydrous acetone of times the weight of 2,4-dimethylpyrrole was added as reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Acetone was removed by rotary evaporation. The residue was extracted with ethyl acetate, dried with anhydrous sodium sulfate and filtered. The organic solvent was removed by rotary evaporation, and the residue was purified by column chromatography on silica gel (eluent: petroleum ether and ethyl acetate containing 1% acetic acid). Finally, an orange-red solid product 2 was obtained (yield 83%).

(3) Synthesis of Compound 3: Compound 2 and N-iodosuccinimide were weighed and added to a reactor at a molar ratio of 1:2. Anhydrous acetonitrile of 10 times the weight of N-iodosuccinimide was added as a solvent, and the reaction was continued for 30 hrs in the dark at normal temperature under argon atmosphere. After the reaction, acetonitrile was removed by rotary evaporation. The residue was purified by column chromatography on silica gel (eluent: petroleum ether and dichloromethane containing 1% acetic acid). Finally, a brick red solid product 3 was obtained (yield 90%).

(4) Synthesis of photosensitizer BDP: Compound 3 and 4-(propynyloxy)benzaldehyde were weighed and added to a reactor at a molar ratio of 1:1. Acetic acid and piperidine of 30 times the molar amount of 4-(propynyloxy)benzaldehyde were added, and then acetonitrile of 30 times the weight of 4-(propynyloxy)benzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and The residue was purified by column chromatography on silica gel (eluent: petroleum ether and dichloromethane containing 1% acetic acid). A blue green solid product BDP was obtained (yield 60%).

(5) Synthesis of photosensitizer cap-BDP: Compound diisopropylethyl amine and vanillylamine hydrochloride were weighed and added to reactor at a molar ratio of 3:2. Acetonitrile of 5 times the weight of diisopropylethyl amine was added as a solvent, and stirred for 15 min at 45° C. under argon atmosphere. Then, the reaction system was positioned in ice bath, and cooled to 0° C. At this time, BDP of 0.5 time of the molar amount of diisopropylethyl amine was added to the reactor, and reacted for another 30 min with stirring. The condensing agent 1-ethyl-(3-dimethylaminopropyl)carbodiimide hydrochloride and 4-dimethylaminopyridine of 2 times the molar amount of diisopropylethyl amine were slowly added to the reaction system, and reacted in an ice bath for another 30 hrs with stirring. After the reaction, N,N-dimethylformamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: ethyl acetate and dichloromethane). A dark green product cap-BDP was obtained (yield 70%).

### Example 4

This example involves the preparation of a compound having a structural formula below:

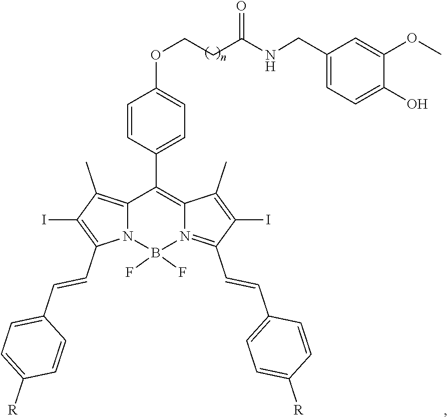

where n=4, and R is methyl.

The compound is prepared through a synthesis route as shown in FIG. 1, and the process comprises specifically the following steps:

(1) Synthesis of Compound 1: p-hydroxybenzaldehyde, 6-bromohexanoic acid and sodium carbonate were weighed and added to a reactor at a molar ratio of 1:2:2. Then acetonitrile of 5 times the weight of 6-bromohexanoic acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with cold acetonitrile to obtain a white crude product. The white crude product was dissolved in ultrapure water, and then neutralized with 4 mol/L hydrochloric acid to obtain a white precipitate. The precipitate was freeze dried, to obtain Compound 1 (yield 80%).

(2) Synthesis of Compound 2: Compound 2,4-dimethylpyrrole and Compound 1 were weighed and added to a reactor at a molar ratio of 2:1. Then, anhydrous acetonitrile of 10 times the weight of 2,4-dimethylpyrrole was added as a reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Acetonitrile was removed by rotary evaporation. The residue was extracted with ethyl acetate, dried with anhydrous sodium sulfate and filtered. The organic solvent was removed by rotary evaporation, and the residue was purified by column chromatography on silica gel (eluent: petroleum ether and ethyl acetate containing 1% acetic acid). Finally, an orange-red solid product 2 was obtained (yield 60%).

(3) Synthesis of Compound 3: Compound 2 and N-iodosuccinimide were weighed and added to a reactor at a molar ratio of 1:2. Anhydrous tetrahydrofuran of times the weight of N-iodosuccinimide was added as a solvent, and the reaction was continued for 30 hrs in the dark at normal temperature under argon atmosphere. After the reaction, tetrahydrofuran was removed by rotary evaporation, and the residue was purified by column chromatography on silica gel (eluent: petroleum ether and dichloromethane containing 1% acetic acid). Finally, a brick red solid product 3 was obtained (yield 90%).

(4) Synthesis of photosensitizer BDP: Compound 3 and p-methylbenzaldehyde were weighed and added to a reactor at a molar ratio of 1:1. Piperidine acetate of 30 times the molar amount of p-methylbenzaldehyde was added, and then acetonitrile of times the weight of p-methylbenzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: 1% acetic acid in dichloromethane and methanol). A blue green solid product BDP was obtained (yield 65%).

(5) Synthesis of photosensitizer cap-BDP: Compound diisopropylethyl amine and vanillylamine hydrochloride were weighed and added to reactor at a molar ratio of 3:2. N,N-dimethyl formamide of 5 times the weight of diisopropylethyl amine was added as a solvent, and stirred for 15 min at 45° C. under argon atmosphere. Then, the reaction system was positioned in ice bath, and cooled to 0° C. At this time, BDP of time of the molar amount of diisopropylethyl amine was added to the reactor, and reacted for another 30 min with stirring. The condensing agent dicyclohexyl carbodiimide and 4-dimethylaminopyridine of 2 times the molar amount of diisopropylethyl amine were slowly added to the reaction system, and reacted in an ice bath for another 30 hrs with stirring. After the reaction, N,N-dimethylformamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: dichloromethane and methanol). A dark green product cap-BDP was obtained (yield 65%).

### Example 5

This example involves the preparation of a compound having a structural formula below:

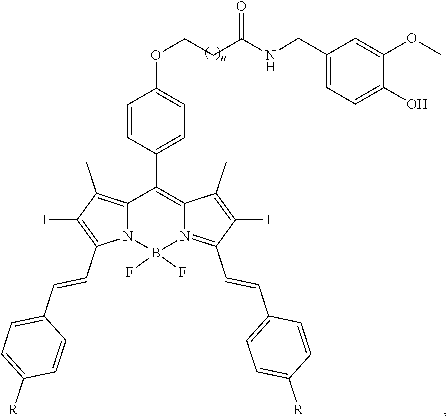

where n=3, and R is t-butoxy.

The compound is prepared through a synthesis route as shown in FIG. 1, and the process comprises specifically the following steps:

(1) Synthesis of Compound 1: p-hydroxybenzaldehyde, 5-bromovaleric acid and sodium carbonate were weighed and added to a reactor at a molar ratio of 1:2:2. Then, acetonitrile of 10 times the weight of 5-bromovaleric acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with cold acetonitrile to obtain a white crude product. The white crude product was dissolved in ultrapure water, and then neutralized with 4 mol/L hydrochloric acid to obtain a white precipitate. The precipitate was freeze dried, to obtain Compound 1 (yield 65%).

(2) Synthesis of Compound 2: Compound 2,4-dimethylpyrrole and Compound 1 were weighed and added to a reactor at a molar ratio of 2:1. Anhydrous dimethyl sulfoxide of 10 times the weight of 2,4-dimethylpyrrole was added as reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Dimethyl sulfoxide was removed by rotary evaporation. The residue was extracted with ethyl acetate, dried with anhydrous sodium sulfate and filtered. The organic solvent was removed by rotary evaporation, and the residue was purified by column chromatography on silica gel (eluent: petroleum ether and ethyl acetate containing 1% acetic acid). Finally, an orange-red solid product 2 was obtained (yield 53%).

(3) Synthesis of Compound 3: Compound 2 and N-iodosuccinimide were weighed and added to a reactor at a molar ratio of 1:2. Anhydrous acetone of 10 times the weight of N-iodosuccinimide was added as a solvent, and the reaction was continued for 30 hrs in the dark at normal temperature under argon atmosphere. After the reaction, tetrahydrofuran was removed by rotary evaporation, and the residue was purified by column chromatography on silica gel (eluent: petroleum ether and dichloromethane containing 1% acetic acid). Finally, a brick red solid product 3 was obtained (yield 87%).

(4) Synthesis of photosensitizer BDP: Compound 3 and t-butoxybenzaldehyde were weighed and added to a reactor at a molar ratio of 1:1. Piperidine acetate of 30 times the molar amount of t-butoxybenzaldehyde was added, and then acetonitrile of times the weight of t-butoxybenzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: 1% acetic acid in dichloromethane and methanol). A blue green solid product BDP was obtained (yield 65%).

(5) Synthesis of photosensitizer cap-BDP: Compound BDP was weighed and added to a reactor at a molar ratio of 3:1 to vanillylamine hydrochloride. 4 was dissolved in N,N-dimethyl formamide of 50 times weight. Then 2-(7-azabenzotriazole)-N,N,N′,N′-tetramethyluronium hexafluorophosphate of 3 times molar amount of vanillylamine hydrochloride and 4-dimethylaminopyridine of 1 time molar amount of vanillylamine hydrochloride were added, and stirred for half an hour in an ice bath. Vanillylamine hydrochloride was added, and stirred for 12 hrs at room temperature under nitrogen atmosphere. After the reaction, N,N-dimethylformamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: dichloromethane and methanol). A dark green product cap-BDP was obtained (yield 65%).

### Example 6

This example involves the preparation of a compound having a structural formula below:

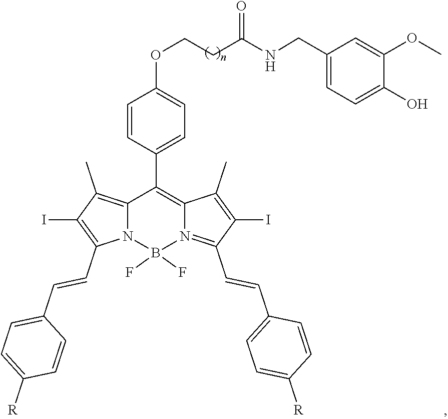

where n=3, and R is ethoxy.

The compound is prepared through a synthesis route as shown in FIG. 1, and the process comprises specifically the following steps:

(1) Synthesis of Compound 1: p-hydroxybenzaldehyde, 5-bromovaleric acid and sodium carbonate were weighed and added to a reactor at a molar ratio of 1:2:2. Then, acetonitrile of 10 times the weight of 5-bromovaleric acid was added as a reaction solvent to the reactor. Finally, a small amount of catalyst benzo-18-crown-6-ether was added, and refluxed for 12 hrs under argon atmosphere. After the reaction, the round-bottomed flask was positioned in an ice bath, and a white precipitate was produced. The resulting precipitate was filtered, and washed with cold acetonitrile to obtain a white crude product. The white crude product was dissolved in ultrapure water, and then neutralized with 4 mol/L hydrochloric acid to obtain a white precipitate. The precipitate was freeze dried, to obtain Compound 1 (yield 81%).

(2) Synthesis of Compound 2: Compound 2,4-dimethylpyrrole and Compound 1 were weighed and added to a reactor at a molar ratio of 2:1. Anhydrous dimethyl sulfoxide of 10 times the weight of 2,4-dimethylpyrrole was added as reaction solvent. 3-5 drops of trifluoroacetic acid were added to the flask, and reacted for 24 hrs at normal temperature under argon atmosphere. Subsequently, 2,3-dichloro-5,6-dicyano-p-benzoquinone of the same molar amount to Compound 1 was added to the reaction system and further reacted for 24 hrs in the reactor. Then, a triethyl amine solution and a boron trifluoride etherate solution of 10 times the weight of 2,4-dimethylpyrrole were slowly added in an ice bath. The reaction was terminated after 24 hrs. Dimethyl sulfoxide was removed by rotary evaporation. The residue was extracted with ethyl acetate, dried with anhydrous sodium sulfate and filtered. The organic solvent was removed by rotary evaporation, and the residue was purified by column chromatography on silica gel (eluent: petroleum ether and ethyl acetate containing 1% acetic acid). Finally, an orange-red solid product 2 was obtained (yield 53%).

(3) Synthesis of Compound 3: Compound 2 and N-iodosuccinimide were weighed and added to a reactor at a molar ratio of 1:2. Anhydrous tetrahydrofuran of times the weight of N-iodosuccinimide was added as a solvent, and the reaction was continued for 30 hrs in the dark at normal temperature under argon atmosphere. After the reaction, tetrahydrofuran was removed by rotary evaporation, and the residue was purified by column chromatography on silica gel (eluent: petroleum ether and dichloromethane containing 1% acetic acid). Finally, a brick red solid product 3 was obtained (yield 87%).

(4) Synthesis of photosensitizer BDP: Compound 3 and p-ethoxybenzaldehyde were weighed and added to a reactor at a molar ratio of 1:1. Piperidine acetate of 30 times the molar amount of p-ethoxybenzaldehyde was added, and then acetonitrile of times the weight of p-ethoxybenzaldehyde was added as a solvent to the reactor. The reaction was continued for 2 hrs at 80° C. under argon atmosphere. After the reaction, acetonitrile was removed by rotary evaporation. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: 1% acetic acid in dichloromethane and methanol). A blue green solid product BDP was obtained (yield 65%).

(5) Synthesis of photosensitizer cap-BDP: Compound BDP was weighed and added to a reactor at a molar ratio of 3:1 to vanillylamine hydrochloride. 4 was dissolved in N,N-dimethyl formamide of 50 times weight. Then 2-(7-azabenzotriazole)-N,N,N′,N′-tetramethyluronium hexafluorophosphate of 3 times molar amount of vanillylamine hydrochloride and 4-dimethylaminopyridine of 1 time molar amount of vanillylamine hydrochloride were added, and stirred for half an hour in an ice bath. Vanillylamine hydrochloride was added, and stirred for 12 hrs at room temperature under nitrogen atmosphere. After the reaction, N,N-dimethylformamide in the reaction system was removed by freeze drying. Then, the residue was extracted with dichloromethane. The organic layer was collected, and dried with anhydrous sodium sulfate and filtered. The organic solvent was removed by rotary evaporation, and the residue was purified by column chromatography on silica gel (eluent: dichloromethane and methanol). A dark green product cap-BDP was obtained (yield 65%).

### Example 7

This example involves the synthesis of the nano-photosensitizer cap-BDP-NPs by self-polymerization of the photosensitizer cap-BDP prepared in Example 1 with the amphiphilic block polymer polyethylene glycol-b-polycaprolactone. The specific process is as follows.

Compound cap-BDP (5 mg) prepared in Example 1 and the amphiphilic block polymer polyethylene glycol-b-polycaprolactone (PEG₁₁₄-b-PCL₆₆, 40 mg) were respectively ultrasonically dissolved in N,N-dimethyl formamide (DMF, 500 mL). After complete dissolution, the cap-BDP solution was added to the PEG₁₁₄-b-PCL₆₆ solution and ultrasonicated for 15 min. Then, 4.2 mL of deionized water was slowly added dropwise to the mixed solution, and ultrasonicated for 15 min. The mixed aqueous solution was dripped by a dropper to a dialysis bag (molecular weight: 3500 KDa), and dialyzed to remove the impurities. 2, 4, 6, 12, 24 hrs after the dialysis, the dialyzing medium was refreshed, where the dialyzing medium was deionized water. 48 hrs after dialysis, the above liquid was transferred to an ultrafiltration tube (3500 KDa), and ultrafiltered and centrifuged (3000 rpm) for 15 min. The upper layer of transparent liquid in the ultrafiltration tube is the nano-photosensitizer cap-BDP-NPs.

Moreover, other amphiphilic block polymers were also used in the present invention, including: polyethylene glycol-polyglutamic acid, poly2-(diisopropylamino)ethyl methacrylate, and polyethylene glycol-polybenzyl glutamate, to replace polyethylene glycol-b-polycaprolactone, with which nano-micelle cap-BDP-NPs can also be prepared.

Performance Test

(1) Tests by UV-Vis Absorption Spectroscopy and Fluorescence Emission Spectroscopy

The free compound cap-BDP prepared in Example 1 and nano-micelle cap-BDP-NPs were tested by UV-Vis absorption spectroscopy and fluorescence emission spectroscopy. The specific operations were as follows.

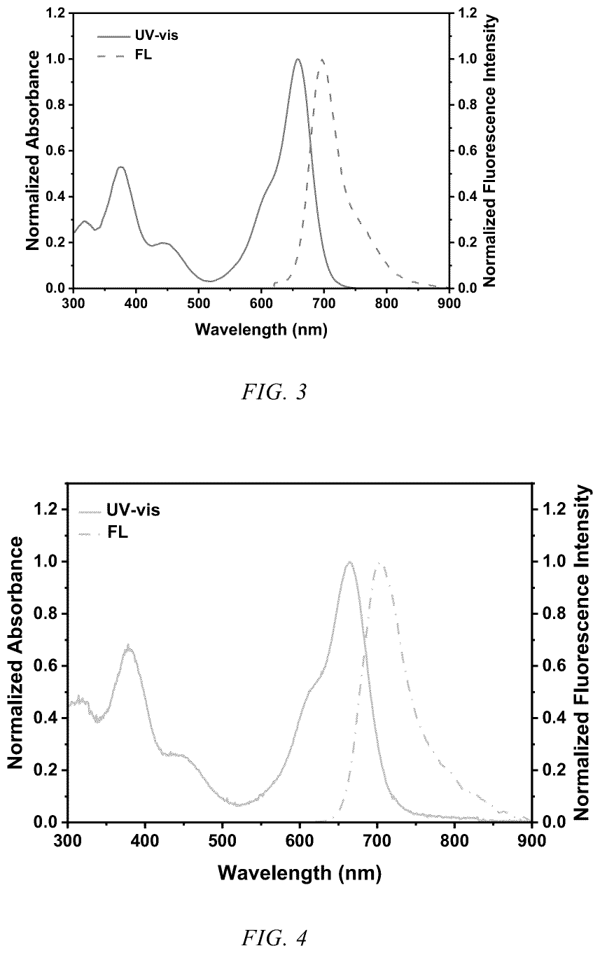

cap-BDP and nano-micelle cap-BDP-NPs were respectively formulated in N,N-dimethyl formamide and ultrapure water to give a 10 mg mL⁻¹ solution. The solution was transferred to a cuvette, and tested by a UV-Vis spectrophotometer and fluorescence spectrophotometer. The test results are shown in FIGS. 3 and 4. The maximum absorption wavelength of cap-BDP is at 660 nm, and the maximum absorption wavelength of nano-micelle cap-BDP-NPs is at 665 nm. The half-peak width becomes wider, indicating J-type aggregation. When the excitation wavelength is fixed at 600 nm, the maximum emission wavelength of cap-BDP is at 692 nm, and the maximum emission wavelength of cap-BDP-NPs is at 702 nm.

(2) Test of Singlet Oxygen Quantum Yield

The singlet oxygen quantum yields of the photosensitizer cap-BDP prepared in Example 1 and commercially available ZnPc were tested under irradiation. The specific operations were as follows.

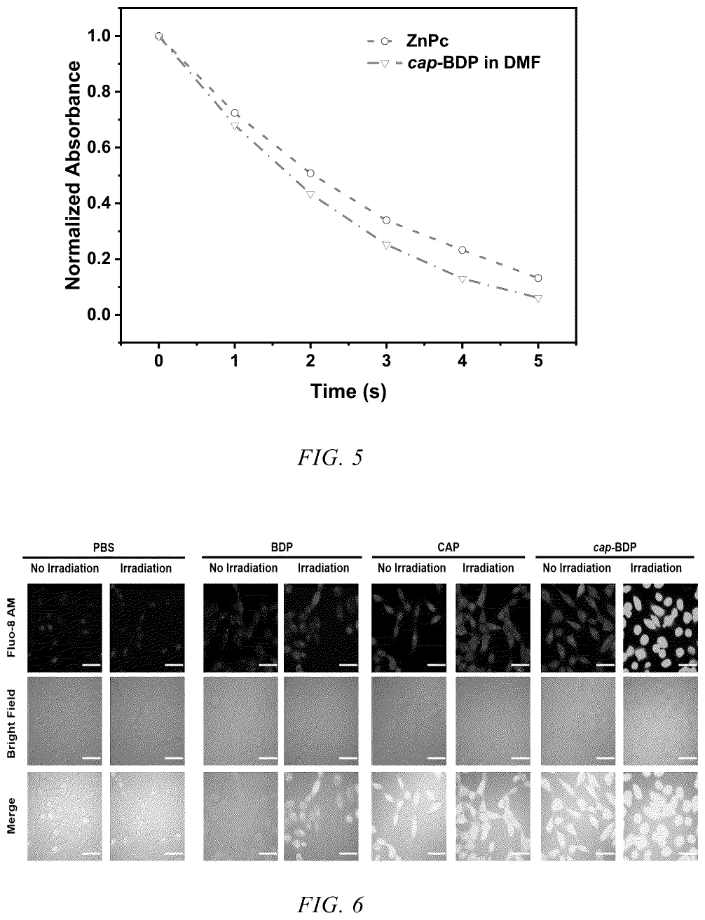

A solution of ZnPc or cap-BDP in N,N-dimethyl formamide was formulated respectively. Each 2.97 mL of the sample solution was added with 30 mL of a DPBF solution (800.0 mg mL⁻¹), mixed uniformly, and transferred to a quartz cuvette. The sample was irradiated with LED light (660 nm, 20 mW cm⁻²), and the absorbance at 415 nm was recorded 0, 1, 2, 3, 4, and 5 s after irradiation. The changes of absorbance was plotted to obtain broken-line diagram. As shown in FIG. 5, cap-BDP and cap-BDP-NPs show a powerful singlet oxygen production ability under irradiation. The singlet oxygen quantum yield of cap-BDP is calculated to be 0.73 with ZnPc (1) as a reference.

(3) Test of Calcium Regulation Ability

The calcium regulation ability of the photosensitizer cap-BDP prepared in Example 1 was tested at the cellular level. The specific operations were as follows.

A high-glucose DMEM cell culture medium containing 10% fetal bovine serum and 5% double antibodies (the following media all contained 10% fetal bovine serum and 5% double antibodies) was prepared. Triple negative breast cancer 4T1 cells in logarithmic growth phase were seeded in a Confocal cell culture dish at a density of 1.0×10⁴ cells/well, and incubated in a cell incubator (37° C., 5% CO₂) for 12 hrs. An irradiation and non-irradiation group were set, and 5.0 μg mL⁻¹ capsaicin (CAP), 1.0 mL BDP and cap-BDP solution (where the BDP and cap-BDP solution contained 1% DMSO) were added respectively. The cells were incubated in an incubator for another 12 hrs, and the culture medium was changed after incubation. The irradiation group was irradiated with LED light (660 nm, 20 mW cm⁻²) for 15 min (without treatment in the non-irradiation group). After irradiation, the culture medium was discarded. The cells were rinsed three times with PBS, and the rinsed cells were stained (10 min) with a fluorescent dye Fluo-8 AM (50.0 μmol L⁻¹, 1.0 mL) for intracellular calcium. After staining, the cells were rinsed with PBS, and observed by laser confocal microscopy.

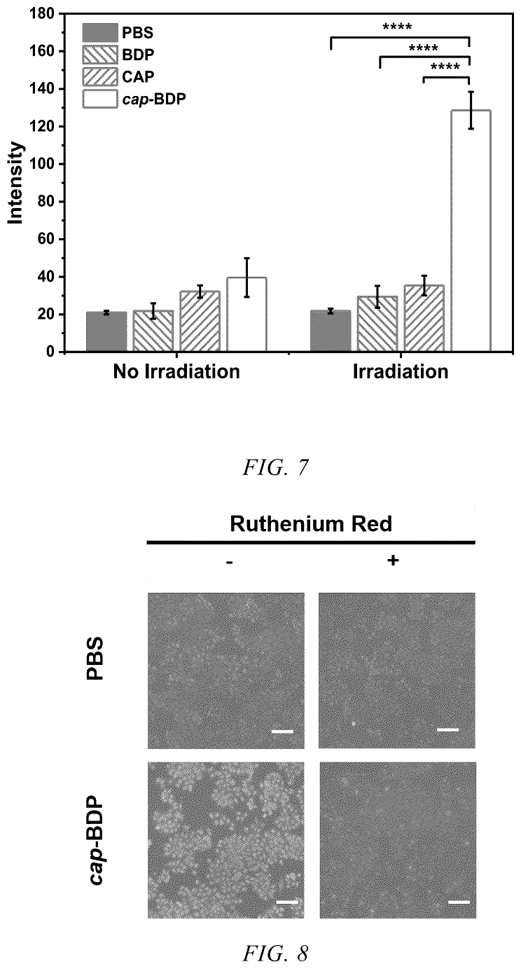

As shown in FIG. 6, the fluorescence intensity of the PBS group does not change significantly with or without irradiation, indicating that irradiation alone cannot cause the change of intracellular calcium concentration. The fluorescence intensity is slightly increased in the photosensitizer BDP group without medication with capsaicin, after irradiation. Compared with the PBS and BDP groups, the CAP group shows intense green fluorescence under non-irradiation and irradiation conditions; however, the fluorescence intensity does not change obviously before and after irradiation. It shows that CAP can effectively activate TRPV1 protein and cause calcium influx, and irradiation can not improve the calcium regulation ability of CAP. The cap-BDP group modified with capsaicin has significantly increased fluorescence intensity after irradiation that is four times the intensity before irradiation (FIG. 7). Moreover, the fluorescence intensity of the cap-BDP group is slightly higher than that of the CAP group under non-irradiation conditions.

According to the above experimental results, similar to CAP, the photosensitizer cap-BDP modified with capsaicin prepared in the present invention can effectively activate TRPV1 protein and change the intracellular calcium concentration. Moreover, light irradiation can effectively improve the calcium regulation ability of the photosensitizer cap-BDP, and TRPV1 protein is further activated by singlet oxygen (¹O₂) to increase the intracellular calcium concentration.

(4) Test of Calcium Channel Type

The calcium channel type of the photosensitizer cap-BDP prepared in Example 1 was tested at the cellular level. The specific operations were as follows.

Triple negative breast cancer 4T1 cells in logarithmic growth phase were seeded in a 12-well plate at a density of 2.0×10⁴ cells/well, and incubated in an incubator (37° C., 5% CO₂) for 12 hrs. The same number of MCF-7 cells were seeded in a 12-well plate, added with a high-glucose DMEM medium containing the TRPV1 inhibitor Ruthenium Red (100.0 μmol L⁻¹, 1.0 mL), and incubated in a cell incubator (37° C., 5% CO₂) for 12 hrs. After incubation, the culture medium was discarded. The cells were rinsed three times with PBS, then added with a cap-BDP solution (5.0 μs mL⁻¹, 1.0 mL), and incubated in an incubator for 12 hrs. The culture medium was changed after incubation. The sample was irradiated with LED light (660 nm, 20 mW^{cm-2}) for 15 min. After irradiation, the culture medium was discarded. The cells were rinsed three times with PBS, and the rinsed cells were stained (10 min) with a fluorescent probe Fluo-8 AM (50.0 μmol L⁻¹, 1.0 mL) for calcium. After staining, the cells were washed three times with PBS, and observed under inverted fluorescence microscope.

As shown in FIG. 8, the cap-BDP group without treatment with Ruthenium Red shows intense green fluorescence, and the cap-BDP group treated with Ruthenium Red merely shows weak green fluorescence. This indicates that cap-BDP can stimulate the opening of TRPV1 channel. After Ruthenium Red is added, the TRPV1 channel is suppressed, and calcium influx is hindered. As a result, the intensity of green fluorescence decreases significantly, indicating that the calcium channel opened by cap-BDP after irradiation is TRPV1 channel.

(5) Cytotoxicity Test

The cytotoxicity of the photosensitizer BDP and cap-BDP prepared in Example 1 under irradiation and non-irradiation conditions and in the presence of external calcium ions. The specific operations were as follows.

Cytotoxicity in the absence of external calcium source: Triple negative breast cancer 4T1 cells in logarithmic growth phase were seeded in a 96-well plate at a density of 8.0×10⁴, and incubated in a cell incubator (37° C., 5% CO₂) for 12 hrs. An irradiation and non-irradiation group were set, and a BDP and cap-BDP solution were respectively added. Six replicate wells were set for each concentration, and the concentrations were 10.0, 5.0, 2.5, 1.25, 0.62, 0.36, and 0.18 μg mL⁻¹ respectively (100.0 μL each well). The cells were incubated in an incubator for another 24 hrs, and the culture medium was changed after incubation. The irradiation group was irradiated with LED light (660 nm, 20 mW cm⁻²) for 15 min (without treatment in the non-irradiation group) and incubated for 24 hrs. After incubation, a MTT solution (5.0 mg mL⁻¹, 20.0 μL) was added to each well, and the cells were incubated in the incubator for another 4 hrs. After the solution was removed, dimethyl sulfoxide was added (150.0 μL) and shaken for 10 min. Finally, the absorbance (OD) of the cell sample at 490 nm was detected on a microplate reader and calculated.

Cytotoxicity in the presence of external calcium source: Triple negative breast cancer 4T1 cells in logarithmic growth phase were seeded in a 96-well plate at a density of 8.0×10⁴ cells/well, and incubated in a cell incubator (37° C., 5% CO₂) for 12 hrs. A BDP and cap-BDP solution were respectively added. Six replicate wells were set for each concentration, and the concentrations were 10.0, 5.0, 2.5, 1.25, 0.62, 0.36, and 0.18 μg mL⁻¹ respectively (100.0 μL each well). The cells were incubated in an incubator for another 12 hrs, and After incubation, the culture medium was changed (containing 60.0 μs mL⁻¹ CaCl₂), 100.0 μL). The sample was irradiated with LED light (660 nm, 20 mW cm⁻²) for 15 min and incubated for 12 hrs. After incubation, a MTT solution (5.0 mg mL⁻¹, 20.0 μL) was added to each well, and the cells were incubated in the incubator for another 4 hrs. After the solution was removed, dimethyl sulfoxide was added (150.0 μL) and shaken for 10 min. The absorbance (OD) of the sample at 490 nm was detected on a microplate reader and calculated.

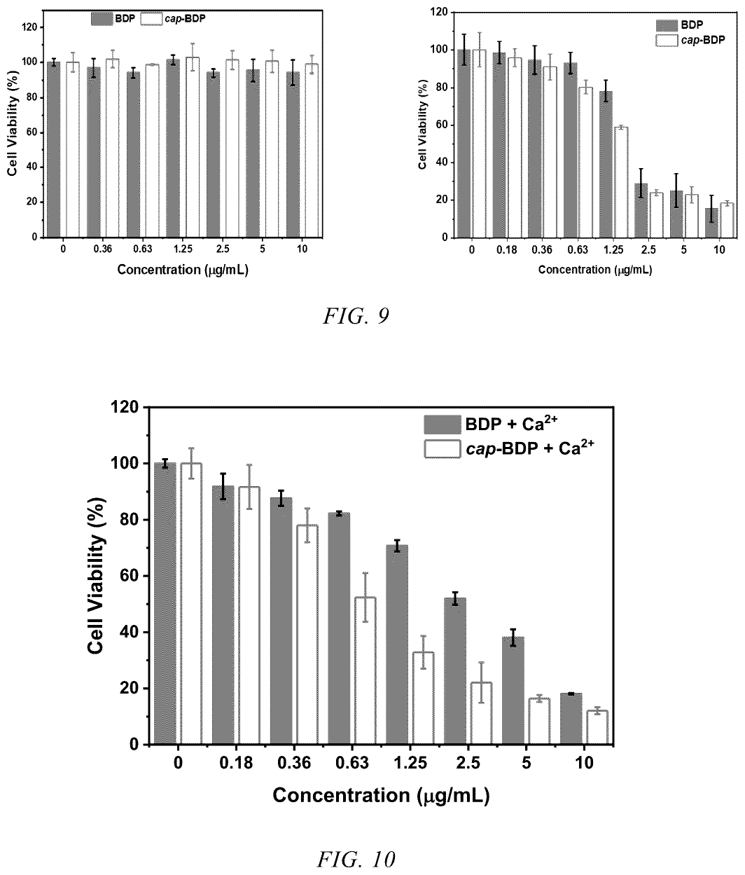

In the absence of an external calcium source, the test results for cytotoxicity of the photosensitizers BDP and cap-BDP on 4T1 cells under irradiation and non-irradiation conditions are as shown in FIG. 9 (the left panel shows the test result under non-irradiation conditions and the right panel shows the test result under irradiation conditions). As can be seen, under non-irradiation conditions, cap-BDP shows no obvious toxicity to 4T1 cells; and under irradiation conditions, cap-BDP and BDP both show strong phototoxicity, with an IC₅₀ value of 1.3±0.1 μg mL⁻¹ and 1.4±0.1 μg mL⁻¹ respectively.

In the presence of a calcium source, the test results for phototoxicity of the photosensitizers BDP and cap-BDP on triple negative breast cancer 4T1 cells are shown in FIG. 10. After calcium is added, the IC₅₀ value of the photosensitizer BDP for 4T1 cells has no significant change, and the IC₅₀ value of the photosensitizer cap-BDP for 4T1 cells decreases from 1.3±0.1 μg mL⁻¹ to 0.6±0.1 μg mL⁻¹. It shows that the photosensitizer cap-BDP not only has good photodynamic activity, but also can further induce apoptosis by causing calcium influx.

(6) Particle Size Distribution and Morphology Characterization of Cap-BDP-NPs

The particle size distribution and morphology of the nano-photosensitizer cap-BDP-NPs prepared in Example 7 were tested. The specific operations were as follows.

Particle size distribution (DLS): A freshly prepared nano-photosensitizer cap-BDP-NPs solution was diluted with ultrapure water to 30.0 μg mL⁻¹ 1.5 mL was added into a particle size test dish, and the particle size distribution was measured by dynamic light scattering (DLS). The sample was tested three times, and each test included 11 rounds.

Morphology characterization (TEM): 10.0 μL of nano-photosensitizer cap-BDP-NPs solution was dripped on to a copper screen, naturally dried in an electronic moisture-proof drying box, and observed by scanning by transmission electron microscopy (TEM, 200 kV).

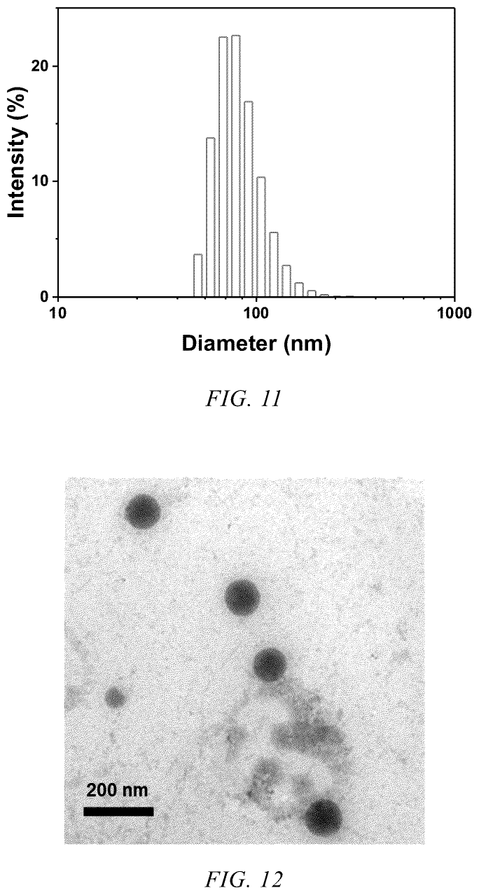

The results are shown in FIG. 11. The average hydration particle size of cap-BDP-NPs in ultrapure water is 128±20 nm, and PDI is 0.117. It can be seen from FIG. 12 that cap-BDP-NPs have a regular spherical structure of 106±12 nm. The test results by DLS and TEM show that the nano-photosensitizer prepared in the present invention has appropriate particle size and good dispersibility. The nano-photosensitizer with uniform nano size can be used to test the biological effects in vivo.

(7) Pharmacokinetic Test of Cap-BDP-NPs

The pharmacokinetics of free compound cap-BDP prepared in Example 1 and nano-photosensitizer cap-BDP-NPs prepared in Example 7 in mice was tested. The specific operations were as follows.

3 female BALB/c mice were administered with 125 I-labelled nano-photosensitizer cap-BDP-NPs (400.0 μg mL⁻¹, 40.0 μCi) by tail vein injection.11 time points (5 min, 10 min, 15 min, 30 min, 1 hr, 3 hrs, 6 hrs, 12 hrs, 24 hrs, 48 hrs, and 72 hrs) were set. At each time point, orbital blood was taken and the mice were weighed. The radioactive content, that is, the drug content, in the blood was tested by a gamma immunocounter. Moreover, the cap-BDP group was set and labeled with a radionuclide. The operation was the same as above.

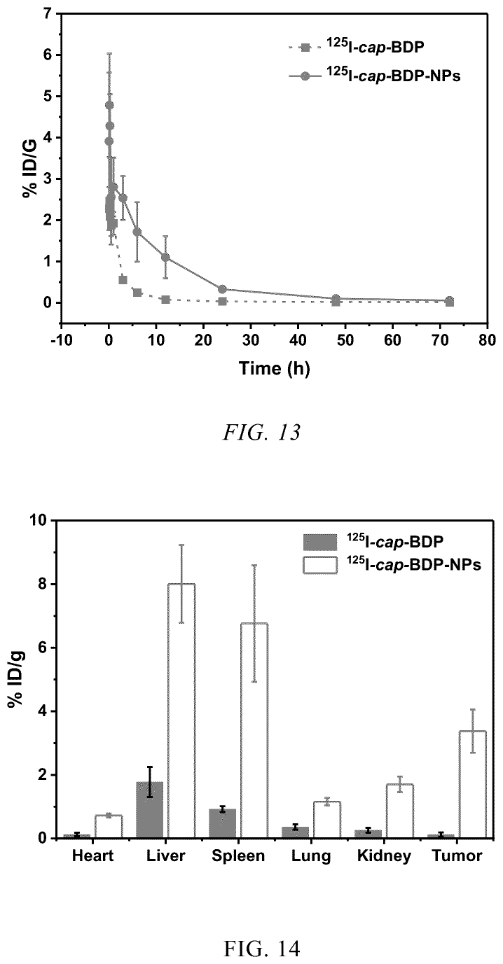

As shown in FIG. 13, the in-vivo clearance half-life of the photosensitizer cap-BDP is 1.1 hrs, and the in-vivo clearance half-life of the nano-photosensitizer cap-BDP-NPs is 15.1 hrs. This result suggests that when the photosensitizer cap-BDP enters mice, it will be metabolized and excreted at a very fast rate; and the nano-photosensitizer cap-BDP-NPs has a good long circulation effect in vivo, and can remain in plasma for a long time, which is conducive to the enrichment of the drug at the tumor site.

(8) Tissue Distribution and SPECT-CT Imaging of Cap-BDP-NPs

The tissue distribution of free compound cap-BDP prepared in Example 1 and nano-photosensitizer cap-BDP-NPs prepared in Example 7 in mice was tested. The specific operations were as follows.

Tissue distribution: 3 tumor bearing female BALB/c mice having a tumor volume of about 200 mm³ were administered with ¹²⁵I-labelled nano-photosensitizer cap-BDP-NPs (400.0 μg mL⁻¹, 60.0 μCi) by tail vein injection.24 hrs later, The mice were dissected and the tissues and organs (heart, liver, spleen, lung, kidney, and tumor) were removed. Each tissue was weighed, and added to a FACS tube. The radioactive content in the tissue was tested by a gamma immunocounter, and quantified by calculation. Moreover, the cap-BDP group was set and labeled with a radionuclide. The operation was the same as above.

SPECT-CT imaging: 1 tumor bearing female BALB/c mouse having a tumor volume of about 200 mm³ was administered with ¹²⁵I-labelled nano-photosensitizer cap-BDP-NPs (400.0 μg mL⁻¹, 100.0 μCi) by tail vein injection.24 hrs later, The mouse were anesthetized and scanned by SPECT-CT imager of small living animals. The performance of targeting and circulation of the nano-photosensitizer cap-BDP-NPs in mice were observed. Moreover, the cap-BDP group was set and labeled with a radionuclide. The operation was the same as above.

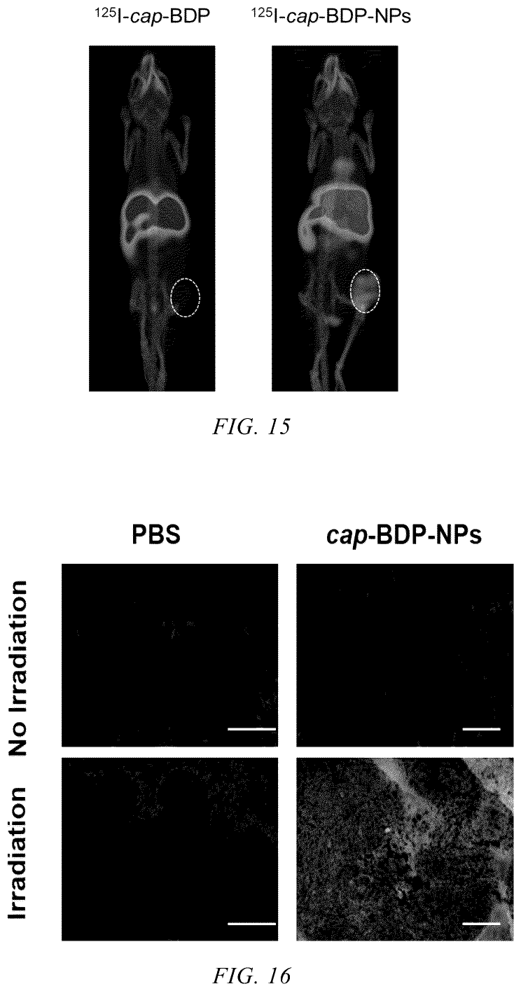

The results of tissue distribution are shown in FIG. 14. The overall distribution of the photosensitizer cap-BDP in mice is low, and the distribution of drug in the tumor is very small, showing that the drug is basically metabolized and excreted from the body. However, the nano-photosensitizer cap-BDP-NPs are highly accumulated at the tumor site, in an amount of about ten times that of 125 I-cap-BDP. The result of SPECT-CT imaging is shown in FIG. 15. The photosensitizer cap-BDP is highly accumulated in liver and bladder 24 hrs after administration, but not in the tumors. The nano-photosensitizer cap-BDP-NPs has a high accumulation concentration at 24 hrs. It shows that the nano-photosensitizer cap-BDP-NPs has good performance of tumor targeting.

(9) Test of In-Vivo Calcium Regulation Ability of Cap-BDP-NPs

The calcium regulation ability of the nano-photosensitizer cap-BDP-NPs prepared in Example 7 in mice was tested. The specific operations were as follows.

6 tumor bearing female BALB/c mice having a tumor volume of about 200 mm 3 were randomized into 2 groups (each having 3 animals), including a PBS group and a cap-BDP-NPs group. PBS, and nano-photosensitizer cap-BDP-NPs solution were respectively administered by tail vein injection (8.0 mg kg⁻¹). 48 hrs later, Fluo-8 AM fluorescent dye (400.0 μmol L⁻¹, 100.0 μL) was injected into the tumor. The tumor sites of the mice were irradiated with LED light (660 nm, 50 mW cm⁻²), for 15 min. After that, the mice were sacrificed by dislocation of cervical vertebrae. The tumor was removed, and immobilized with 4% paraformaldehyde solution in the dark for 24 hrs. Then, the tumor tissue was embedded, and the tumor tissue was sliced by using a kryotome, and observed and photographed under a fluorescence inverted microscope.

As shown in FIG. 16, no obvious green fluorescence is observed in the PBS group, and the cap-BDP-NPs group with irradiation shows obvious green fluorescence, showing that the nano-photosensitizer cap-BDP-NPs can effectively cause calcium influx in the tumor site after irradiation, and the calcium concentration in the tumor tissue is improved.

(10) Test of Inhibition of Cap-BDP-NPs on Tumors

The tissue distribution of free compound cap-BDP prepared in Example 1 and nano-photosensitizer cap-BDP-NPs prepared in Example 7 in mice was tested. The specific operations were as follows.

tumor bearing female BALB/c mice having a tumor volume of about 60 mm 3 were randomized into 6 groups (each having 5 animals), including specifically irradiation/non-irradiation PBS groups, irradiation/non-irradiation cap-BDP groups, and irradiation/non-irradiation cap-BDP-NPs groups. The mice in each group were respectively administered by tail vein injection (8.0 mg kg⁻¹); and 48 hrs after administration, the tumor site of mice was irradiated with LED light (660 nm, 50 mW cm⁻²) for 15 min. Then the changes of tumor volume in mice within 21 days after irradiation were recorded, and a curve of tumor volume vs time was plotted, to evaluate the antitumor effect of the preparation.

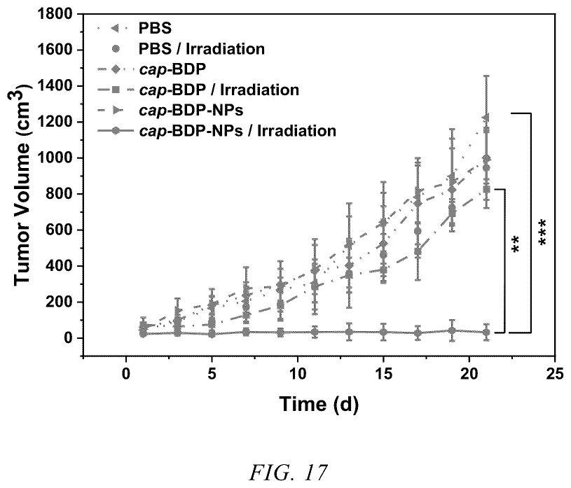

As shown in FIG. 17, it can be known from the tumor inhibition curve that there is no significant difference in tumor growth curves between the irradiation PBS group and non-irradiation PBS group. This shows that the tumor growth cannot be effectively inhibited by light only, and the tumor volume is about 24 times the initial volume. No obvious tumor inhibition effect is produced in the irradiation cap-BDP group, and the tumor volume is about 17 times the initial volume. This may be due to the rapid in-vivo metabolism of the photosensitizer cap-BDP and less accumulation in the tumor tissue. The irradiation cap-BDP-NPs group shows significant tumor inhibition effect, and some tumors are ablated and have no recurrence within 21 days. The above results show that the nano-photosensitizer cap-BDP-NPs can effectively produce reactive oxygen species after irradiation, and cause the calcium influx at the tumor site in mice, so as to achieve a synergistic therapeutic effect. Therefore, it can effectively inhibit the tumor growth in mice, and has a superior in-vivo therapeutic effect.

The above-described embodiments are merely preferred embodiments for the purpose of fully illustrating the present invention, and the scope of the present invention is not limited thereto. Equivalent substitutions or modifications can be made by those skilled in the art based on the present invention, which are within the scope of the present invention as defined by the claims. The scope of the present invention is defined by the appended claims.

1. A capsaicin-derived photosensitizer, having a general structural formula shown below: 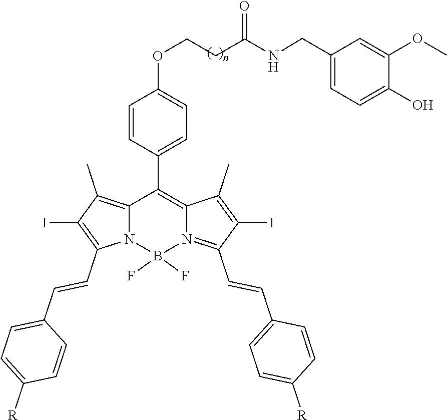
wherein n is any integer from 1 to 10;
R is selected from hydrogen, C1-C8 alkyl, and C1-C8 alkoxy.

2. A method for preparing a capsaicin-derived photosensitizer according to claim 1, comprising the following steps: under an inert atmosphere,
(1) reacting Compound of Formula (I) with 2,4-dimethylpyrrole in the presence of trifluoroacetic acid and an organic solvent, then adding an oxidant for further reaction, adding boron trifluoride etherate and an organic amine to the system after the reaction, and reacting to obtain Compound of Formula (II);
(2) reacting Compound of Formula (II) with an iodination reagent in the presence of an organic solvent, to obtain Compound of Formula (III);
(3) reacting Compound of Formula (III) with Compound of Formula (IV) in the presence of acetic acid, piperidine and an organic solvent, to obtain Compound of Formula (V); and
(4) reacting Compound of Formula (V) with vanillylamine or a salt thereof in the presence of a condensing agent and an organic solvent, to obtain the capsaicin-derived photosensitizer;
Wherein the structures of Formulas (I)-(V) are shown below: 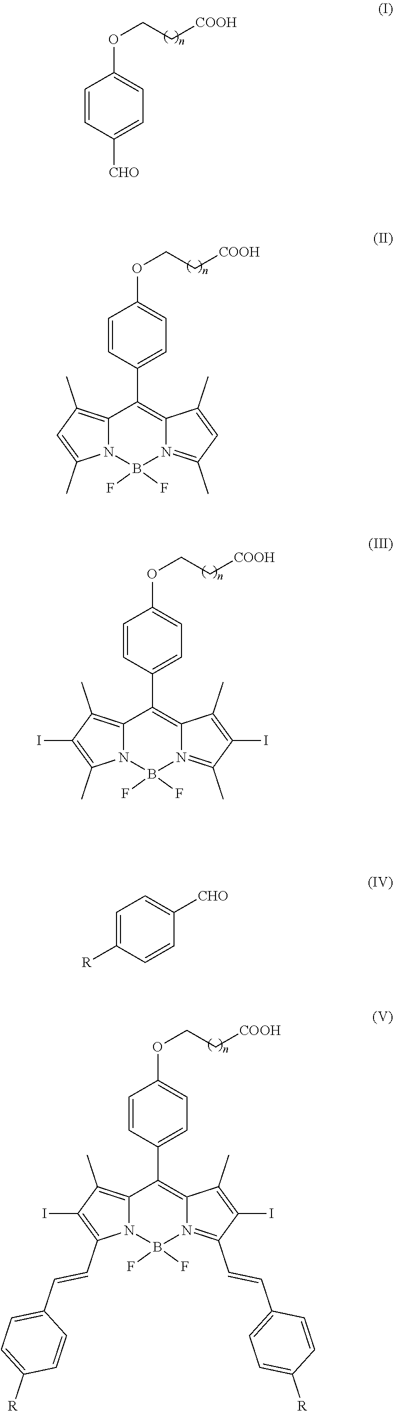
where n is any integer from 1 to 10, R is selected from hydrogen, C1-C8 alkyl, and C1-C8 alkoxy.

3. The preparation method according to claim 2, wherein in Step (1), the oxidant is 2,3-dichloro-5,6-dicyano-p-benzoquinone or selenium dioxide; and the organic amine is triethyl amine or diisopropylethyl amine.

4. The preparation method according to claim 2, wherein in Step (2), the iodination reagent is N-iodosuccinimide, elemental iodine or N-iodosaccharin.

5. The preparation method according to claim 2, wherein in Step (4), the condensing agent is (1-cyano-2-ethoxy-2-oxoethylideneaminooxy)dimethylamino-morpholin-carbonium hexafluorophosphate, dicyclohexyl carbodiimide, 1-ethyl-(3-dimethylaminopropyl)carbodiimide hydrochloride or 2-(7-azabenzotriazole)-N,N,N′,N′-tetramethyluronium hexafluorophosphate.

6. The preparation method according to claim 2, wherein in Step (4), the reaction is carried out in the presence of an acid acceptor and/or a catalyst, in which the acid acceptor is diisopropylethyl amine or triethyl amine, and the catalyst is 4-dimethylaminopyridine.

7. A nano-photosensitizer, formed by self-assembly of the capsaicin-derived photosensitizer according to claim 1 with an amphiphilic block polymer in water.

8. The nano-photosensitizer according to claim 7, wherein the amphiphilic block polymer is selected from the group consisting of polyethylene glycol-b-polycaprolactone, polyethylene glycol-polyglutamic acid, poly2-(diisopropylamino)ethyl methacrylate, and polyethylene glycol-polybenzyl glutamate.

9. A method for preparing the nano-photosensitizer according to claim 7, comprising: dissolving the capsaicin-derived photosensitizer and an amphiphilic block polymer in an organic solvent, adding water to the obtained solution, and dialyzing to obtain the nano-photosensitizer; wherein the dialyzing medium for dialysis is water.

10. Use of the nano-photosensitizer according to claim 7 in the preparation of drugs for light-induced treatment of triple negative breast cancer.
